(19) United States
(12) Patent Application Publication
(10) Pub. No.: US 2020/0015882 A1
(43) Pub. Date: Jan. 16, 2020

(54) Integrated Electrode

(71) Applicant: BEIJING MED ZENITH MEDICAL SCIENTIFIC CO., LTD., Beijing (CN)

(72) Inventors: Jian MENG, Beijing (CN); Baoqi XIE, Beijing (CN); Zhiwei MA, Beijing (CN); Danian KE, Beijing (CN); Xiaofang LIU, Beijing (CN)

(21) Appl. No.: 16/335,269

(22) PCT Filed: Dec. 29, 2016
(86) PCT No.: PCT/CN2016/112825

§ 371 Date: Mar. 21, 2019

(30) Foreign Application Priority Data
Sep. 30, 2016 (CN) 201610874702.1

(51) Int. Cl. A61B 18/14; B29C 45/14

(52) U.S. Cl. CPC A61B 18/1445; A61B 2018/00577; B29C 45/14311

(57) ABSTRACT

Some embodiments of the present disclosure provide an integrated electrode, the integrated electrode includes an electrode wire and an electrode base. The electrode wire is provided in a hollowed-out shape, and several through holes are punched on a side face of the electrode wire without changing the appearance and dimension of the electrode wire. The electrode base is formed by an injection molding process, and is integrally connected with the electrode wire by means of a one-time injection molding. During a process of injection molding, plastic fluid can flow through the hollowed-out through holes of the electrode wire so as to fix the electrode wire in the electrode base, such that the electrode wire and the electrode base are integrated.

### TECHNICAL FIELD

The present disclosure belongs to a technical field of radiofrequency ablation pincers, and particularly relates to an integrated electrode applied to bipolar radiofrequency ablation pincers.

### BACKGROUND

In a related art, bipolar radiofrequency ablation pincers are a medical instrument applied to cardiothoracic minimally invasive surgeries, the radiofrequency ablation pincers are used for performing ablation isolation of circumferential pulmonary vein orifices at two sides, an ablation line is accurate, complete, and wall-penetrating, and it is an effective method for treating atrial fibrillation at present.

The atrial fibrillation is the most common arrhythmia clinically, according to statistics, there are about 8 million of atrial fibrillation patients in China, and population above 60 years old is a high risk group. The atrial fibrillation may generate many hazards to a human body. During the atrial fibrillation, a frequency of atrium beats is very fast, and irregular, a less serious patient may feel palpitation, shortness of breath, and debilitation, cardiac functions may be damaged more or less, if hypertension or a coronary heart disease is generated at the same time, risks of these heart diseases may be aggravated. The atrial fibrillation may also generate serious complication, such as ischemic stroke caused by falling off of atrial thrombus, the disability, and death may be caused.

The bipolar radiofrequency ablation pincers are used for performing a minimally invasive ablation surgery without extracorporeal circulation, and the surgery is performed in ^{<a} state that heartbeat is not stopped, principal steps are bilateral pulmonary vein isolation, left atrium circumferential pulmonary vein line ablation, epicardium partial denervation and left aurcle excision. The advantage is that surgery injury is small, operation is rapid and accurate, complication is less, and curative effect is good. Paroxysmal atrial fibrillation is used as a major treatment object internationally, and strictly selected persistent and perpetual atrial fibrillation is also included, a total curative ratio within 6 months may reach 91,3%. Surgery time is 2-4 hours generally, and average hospital stay is 3-5 days. After 6 months of the ablation, with regard to a patient of the paroxysmal atrial fibrillation, a sinus rhythm conversion rate is 80-90% (18-20). In addition, along with development of technologies, these surgeries are simpler and simpler, and easy to repeated operation. Compared with a catheter intervention technology, the minimally invasive surgery is one-time treatment, herein, a long-term curative ratio, according to existing abroad experience report, may reach about 90%. Experience of the atrial fibrillation treatment center of the Beijing Anzhen hospital is as follows: the sinus rhythm on discharge is 81.8% (18/22), and the sinus rhythm in 3 months after the surgery is 90% (9/10).

As shown in FIG. 1 and FIG. 2, a technical scheme of an electrode part of the bipolar radiofrequency ablation pincers in the related art is that an electrode groove 100 (as shown in FIG. 1) is reserved in an injection moulding process of an electrode base, after medical glue is applied in the electrode groove, an electrode wire 101 is pressed into the electrode groove (an electrode groove gluing layer 102 is formed in the electrode groove.), and integrally bonded and fixed with an electrode base 103 (as shown in FIG. 2). In a product working state of the electrode part of this bipolar radiofrequency ablation pincers in the related art, along with rising of a temperature of the electrode wire, bonding capacity of the gluing layer is reduced after hot-melting deformation, the risks of upwarp, ejection from the electrode groove or falling out exist in the electrode wire.

Thus it may be seen that after the bipolar radiofrequency ablation pincers in the related art are used for a long time, a working part easily generates problems of electrode upwarp and falling out, in a high temperature environment. In use, a temperature of a working terminal, namely the electrode part, of the bipolar radiofrequency ablation pincers is gradually ascended along with time, and the risks of upwarp and falling out exist in an electrode which is positioned in the high temperature environment for a long time.

### SUMMARY

Some embodiments of the present disclosure provide an integrated electrode, a technical problem to be solved is how to enable an electrode wire to be integrally with an electrode base, and falling out may not be generated again.

In order to achieve the above purpose, the integrated electrode of some embodiments of the present disclosure includes the electrode wire and the electrode base, the electrode wire is provided in a hollowed-out shape, and several through holes are punched on a side face of the electrode wire without changing an appearance and dimension of the electrode wire. The electrode base is formed by an injection molding process, and is integrally formed with the electrode wire by means of a one-time injection molding.

During a process of injection molding, plastic fluid can flow through the hollowed-out through holes of the electrode wire so as to fix the electrode wire in the electrode base, such that the electrode wire and the electrode base are integrated.

Several glue-coated pits bonded to the electrode wire are provided inside the electrode base, and after the glue-coated pits are fully covered with glue, the fixing of the electrode wire is further guaranteed.

A technical function of the glue-coated pits, in an injection moulding mould, is convenient for fixing the electrode wire in a groove of a mould, and positioning the electrode wire, such that the injection moulding is completed.

The electrode wire includes an inner side electrode wire and an outer side electrode wire,

Some embodiments of the present disclosure have the following advantages: compared with the related art, the integrated electrode of the present disclosure is capable of integrally fixing the electrode wire and the electrode base in a mode of the most fastness, compared with an existing fixing process, the integrated electrode is safer, more stable, and more effective, and capable of minimizing the risk of upwarp, ejection and falling out of electrode wire. The plastic is injected into the through holes on the electrode wire, so as to integrally connect the electrode wire and the electrode base, such that falling out does not occur.

### BRIEF DESCRIPTION OF THE DRAWINGS

FIG. 1 is a structure schematic diagram of an electrode part of bipolar radiofrequency ablation pincers in the related art;

FIG. 2 is a profile diagram of the electrode part as shown in FIG. 1;

FIG. 3 is a structure schematic diagram of a hollow-out electrode wire of the integrated electrode according to an embodiment of the present disclosure;

FIG. 4 is a structure schematic diagram of glue-coated pits of the integrated electrode according to an embodiment of the present disclosure;

FIG. 5 is a stereostructure schematic diagram of the integrated electrode according to an embodiment of the present disclosure; and

FIG. 6 is a profile diagram of the integrated electrode according to an embodiment of the present disclosure.

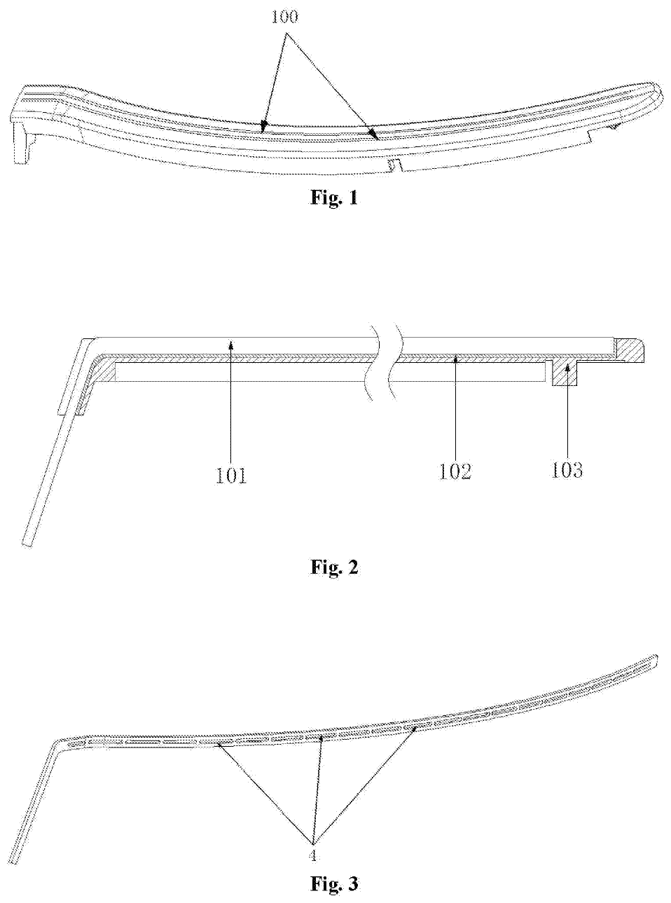

### DETAILED DESCRIPTION OF THE EMBODIMENTS

The following embodiments are used for describing the present disclosure, but not intended to limit a scope of the present disclosure.

As shown in FIG. 3 to FIG. 6, an embodiment of the present disclosure provides an integrated electrode, the integrated electrode includes an electrode wire 1 and an electrode base 2, the electrode wire 1 is provided in a hollowed-out shape, and several through holes 4 are punched on a side face of the electrode wire 1 without changing an appearance and dimension of the electrode wire, the electrode base 2 is formed by an injection molding process, and is integrally formed with the electrode wire 1 by means of a one-time injection molding.

During a process of an injection molding, plastic fluid can flow through the hollowed-out through holes of the electrode wire, so as to fix the electrode wire in the electrode base (as shown in FIG. 3), such that the electrode wire and the electrode base are connected together and forms one integrated structure.

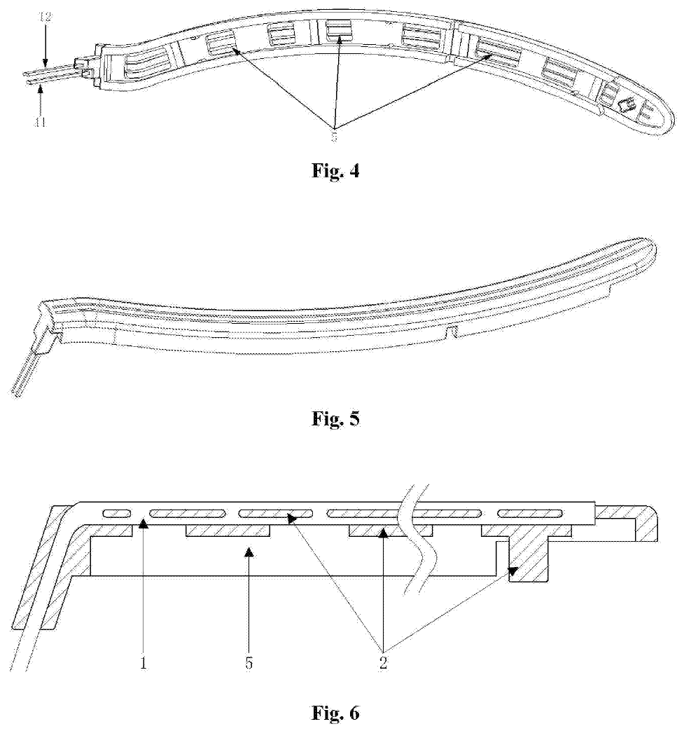

In an exemplary embodiment, as shown in FIG. 4, several glue-coated pits 5 bonded to the electrode wire are provided inside the electrode base, and after the glue-coated pits are fully covered with glue, the fixing of the electrode wire is further guaranteed.

A technical function of the glue-coated pits, in an injection moulding mould, is convenient for fixing the electrode wire in a groove of the mould, and positioning the electrode wire, such that the injection moulding is completed.

In an exemplary embodiment, as shown in FIG. 4, the electrode wire includes an inner side electrode wire 11 and an outer side electrode wire 12.

A key point of some embodiments of the present disclosure is that the electrode wire is provided in the hollowed-out shape, namely several through holes (as shown in FIG. 3) are punched on the side face of the electrode wire without changing the appearance and dimension of the electrode wire; and the electrode base is formed by the injection molding process, and is integrally formed with the electrode wire by means of the one-time injection molding. A technology of the scheme is that the plastic fluid injected into a mould cavity of an electrode base may flow through the hollowed-out through holes of the electrode wire so as to fix the electrode wire in the electrode base (as shown in FIG. 6). After the pits are fully covered with the glue, the fixing of the electrode wire is further guaranteed, the technical scheme is capable of integrally fixing the electrode wire and the electrode base in a mode of the most fastness, and is safer, more stable, and more effective than existing fixing processes. The plastic is injected into the above through holes, so as to integrally connect the electrode wire and the electrode base, such that a problem of falling out does not occur.

In an embodiment of the present disclosure, several glue-coated pits bonded to the electrode wire are provided inside the electrode base (as shown in FIG. 4); a function of the glue-coated pits is to conveniently glue at this place, and an upper and lower electrode bases and the electrode wire are bonded to upper and lower edges of a metal together. A technical function of the pits, in an injection moulding mould, is convenient for fixing the electrode wire in a groove of a mould, and positioning the electrode wire, such that the injection moulding is completed.

A technical point of the present disclosure is that the side face of the electrode wire is provided with several through holes, the electrode wire is in the hollowed-out shape (as shown in FIG. 3), and integrally formed with the electrode base by means of the one-time injection molding, several glue-coated pits bonded to the electrode wire are arranged inside the electrode base (as shown in FIG. 4), plastic fluid injected into an electrode base mould cavity may flow through the hollowed-out through holes of the electrode wire so as to fix the electrode wire in the electrode base (as shown in FIG. 6), so the integrated electrode is realized, in use, the pits are internally covered with the glue again, the fixing of the electrode wire may be further guaranteed.

Although the present disclosure is described in detail in the above by using general description and specific embodiments, it is apparent to those skilled in the art that some modifications or improvements may be made on the basis of the present disclosure. Therefore, these modifications or improvements made on the basis without deviating from spirit of the present disclosure shall fall within a scope of protection required by the present disclosure.

What is claimed is:

1. An integrated electrode, wherein the integrated electrode comprises an electrode wire and an electrode base, the electrode wire is provided in a hollowed-out shape, and several through holes are punched on a side face of the electrode wire without changing an, appearance and dimension of the electrode wire, the electrode base is formed by an injection molding process, and is integrally connected with the electrode wire by means of a one-time injection molding.

2. The integrated electrode as claimed in claim 1, wherein during a process of injection molding, plastic fluid can flow through the hollowed-out through holes of the electrode wire so as to fix the electrode wire in the electrode base, such that the electrode wire and the electrode base are integrated.

3. The integrated electrode as claimed in claim 2, wherein several glue-coated pits bonded to the electrode wire are provided inside the electrode base, and after the glue-coated pits are fully covered with glue, fixing of the electrode wire is further guaranteed.

4. The integrated electrode as claimed in claim 3, wherein a technical function of the glue-coated pits, in an injection moulding, mould, is convenient for fixing the electrode wire in a groove of a mould, and positioning the electrode wire, such that the injection moulding is completed.

5. The integrated electrode as claimed in claim 3, wherein the electrode wire includes an inner side electrode wire and an outer side electrode wire.
